(19) United States
(12) Patent Application Publication
(10) Pub. No.: US 2021/0275836 A1
(43) Pub. Date: Sep. 9, 2021

(54) RESPIRATORY PROTECTION MASK

(71) Applicants: MARKUS BINDHAMMER, FRIEDBERG (DE); HOWARD T. BELLIN, NEW YORK, NY (US)

(72) Inventors: MARKUS BINDHAMMER, FRIEDBERG (DE); HOWARD T. BELLIN, NEW YORK, NY (US)

(21) Appl. No.: 16/934,557

(22) Filed: Jul. 21, 2020

(30) Foreign Application Priority Data
Mar. 9, 2020 (EP) EP20020113.5

(51) Int. Cl. A62B 7/10; A41D 13/11; A62B 18/02

(52) U.S. Cl. CPC A62B 7/10; A41D 2400/26; A62B 18/02; A41D 13/1138

(57) ABSTRACT

In a respiratory protection mask with a face mask part (2) and, associated with the latter, an air disinfection device (3) comprising a housing (4) exhibiting an input communicating with the environment, and further an output communicating with the internal area of the face mask part (2) and whose internal area is designed as an air irradiation chamber (16) provided with at least one LED light source (6) emitting UV light, a high user-friendliness is achieved in that the LED light source (6) is arranged on a slide (7) which is detachably receivable in an associated frame (8) provided on the housing side and open towards the air irradiation chamber (16).

The present invention relates to a respiratory protection mask with a face mask part and, associated with the latter, an air disinfection device comprising a housing exhibiting an input communicating with the surrounding, and further an output communicating with the internal area of the face mask part and whose internal area is designed as an air irradiation chamber provided with at least one LED light source emitting UV light.

A respiratory protection mask of this kind is known from WO 2017/014102 A1. Arrangements of this kind serve as prophylaxis against smear and droplet infection. In the known arrangement the LED light source is embedded in a firm part of the housing rendering manufacture of the housing as well as assembly difficult. As a consequence of the arrangement of the LED light source being integrated in the housing, the latter cannot be accessed from the outside, thus rendering maintenance and cleaning difficult. Cleaning the housing, which becomes necessary from time to time, is also difficult. The known arrangement has thus proved to be not sufficiently simple and user-friendly.

On the basis of the foregoing it is the object of the present invention to improve a respiratory protection mask as described initially above in such a way that the disadvantages of the known arrangement are avoided and a simple manufacture and assembly as well as a high user-friendliness is achieved.

According to the present invention, this object is achieved in that the LED light source is arranged on a slide which is detachably receivable in an associated frame provided on the housing side and open towards the air irradiation chamber.

These measures according to the invention advantageously result in a structural separation of the arrangement of the LED light source from the housing and thus provide for simple manufacture of the housing as well as simple assembly and easy access to the LED light source. By actuating the slide the LED light source received thereon can easily be inserted in the housing and/or withdrawn from the latter thus ensuring easy maintenance and cleaning of the LED light source as well as the housing. The latter may simply be designed as a plastics part which for cleaning purposes may be cleaned in a dish washer or a washing machine after the slide has been removed. The same applies to the face mask part which preferably consists of textile material. The slide together with the LED light source can be handled separately during cleaning, so that a correspondingly careful handling of the LED light source will be ensured.

Advantageous embodiments and expedient developments of the main-claim measures will be evident from the sub-claims.

Expediently, the slide may be detachably attachable to the frame by means of an associated snap-on device and exhibit a manually actuatable unlocking element attached to the latter. These measures ensure reliable handling of the slide and simultaneously ensure a reliable hold of the slide in the housing and thus a high degree of trouble-free operation.

In order to further increase operational safety, a push button may be associated with the LED light source which is actuated only when the slide is entirely inserted.

A further expedient measure may consist in the that the housing is fitted with a region in the shape of a cylinder section and an appendage adjoining its jacket in the fashion of a secant, comprising the frame with the input and the output of the air irradiation chamber being associated with the mutually opposite end faces of the housing. These measures result in a compact, clear-cut design of the housing.

In accordance with an alternative embodiment the housing may be of angle-shaped design. This design results in a comparably large bipartite internal area, so that provision may be made for two slides positioned at an angle towards each other. In addition, the housing may be designed in a way to accommodate the symmetry of the face.

Expediently, the frame may be designed as a recess of the appendage associated with the slide comprising guide surfaces, adapted to the configuration of the slide, exhibiting a clear internal configuration, intersected with the air irradiation chamber, with the LED light source, when the slide is inserted, being placed in a region of the slide reaching the region of the recess intersected with the air irradiation chamber. These measures result in simple operation of the slide when the slide is inserted. These measures result in simple operation of the slide.

Expediently, the housing may have two mutually attachable halves, each of them comprising a housing end face. This ensures easy manufacture of the housing as a casting, preferably an injection-moulded part consisting of plastics.

A further, particularly preferable measure may consist in that associated with the input and output of the housing are mutually spaced parallel slats which are tilted towards the housing axis. This ensures that UV radiation which is dangerous for the human eyes and skin cannot issue from the housing and affect the face of the wearer of the respiratory protection mask or the environment. These measures thus favour the use of a LED light source emitting particularly short-wave aggressive UV light which possesses a particularly good germ-killing effect.

Expediently, provision may be made for a LED light source as UVC light with a wave length in the range of 250 nm to 260 nm, in particular of UVC LED emitting 254 nm with a forward voltage of a ≥5 V as well as a radiation power of a ≥100 mW at the current intensity resulting from the corresponding forward voltage and a large radiation angle in the region of 120°.

In order to increase the effect of the LED light source, the internal surface of the light irradiation chamber may be covered with a reflecting metal coating. In the case of a plastics housing a further advantage of the metal coating consists in screening the plastics surface against UV and/or UVC radiation, so that the plastics material is protected against a fast ageing process caused by the said radiation.

As material used for the mirrors provision may be made in an advantageous way for silver compounds having an anti-bacterial effect.

A further, advantageous measure of the main-claim measures may consist in that the slide in the region of a bulge provided in the rear section of the slide in insert direction comprises an electrically conducting bushing connected to each of the LED light sources received on the said slide, such bushing being connectable to a portable energy source via a plug-in cable. These measures permit a mobile use of the respiratory protection mask according to the present invention and thus ensure a high degree of user-friendliness.

A particularly preferable further development may consist in a filter device arranged ahead of the air irradiation device, which removes the particles contained in the air sucked in from outside. Expediently, to facilitate cleaning, the filter device may comprise a filter body consisting of an aerogel and acting together with ejection springs and a security rotary lever.

Advantageously, the LED light source may be arranged in such a manner that its light also shines on the filter. Thus, the breathing air as well as the filter material are simultaneously and continually disinfected.

A further advantageous measure may consist in a cooling device being associated with the LED light source, which comprises a cooling body expediently provided on the slide and communicating with the surrounding. In addition or alternatively hereto provision may be made for the warmth of the LED light source to be used for drying the filter.

Expediently, the bushing may be designed as an USB bushing which is connectable to a portable power bank via an USB cable, These measures permit a particularly long time of use.

Further advantageous embodiments and expedient further developments of the main-claim measures will be evident from the description of an example given below in conjunction with the accompanying drawings, wherein

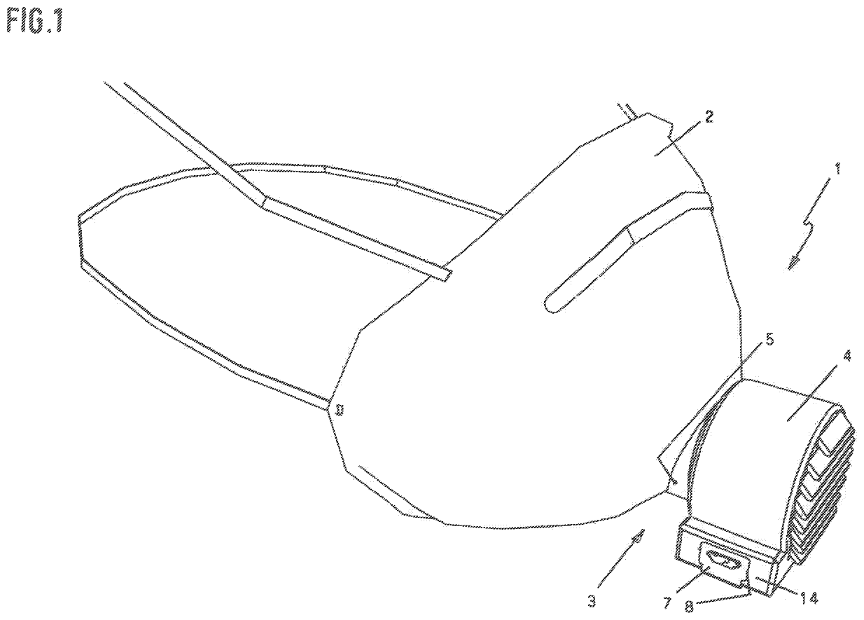

FIG. 1 is a perspective view of a respiratory protection mask according to the invention;

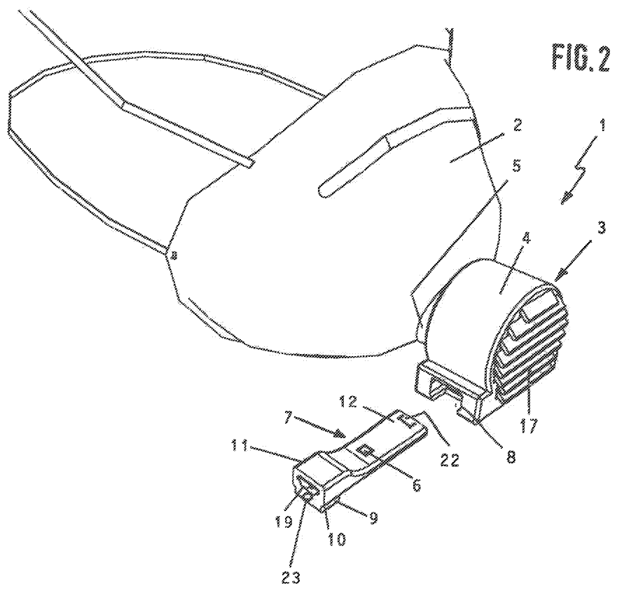

FIG. 2 shows the arrangement according to FIG. 1 with the slide pulled out;

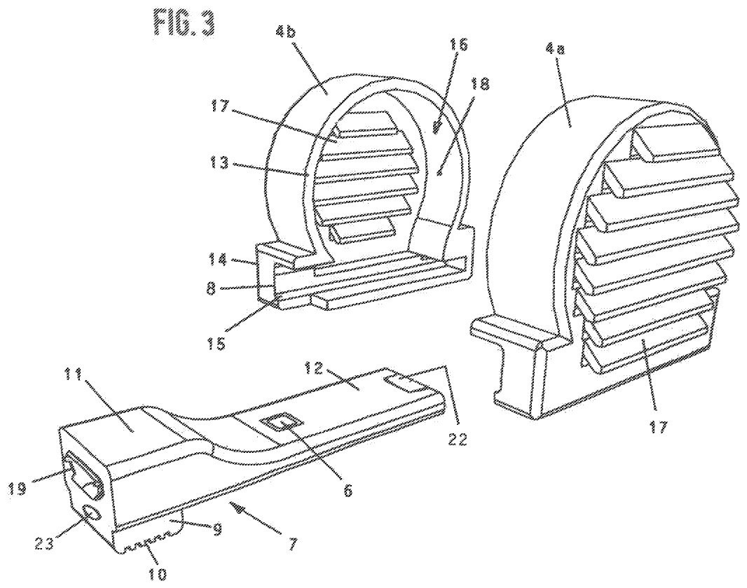

FIG. 3 shows an exploded view of the housing of the arrangement according to FIG. 2 with the slide pulled out;

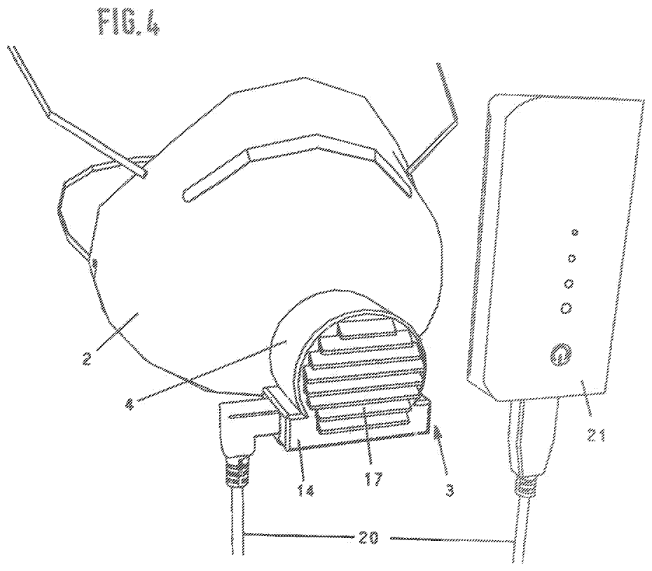

FIG. 4 shows the arrangement according FIG. 1 ready for use;

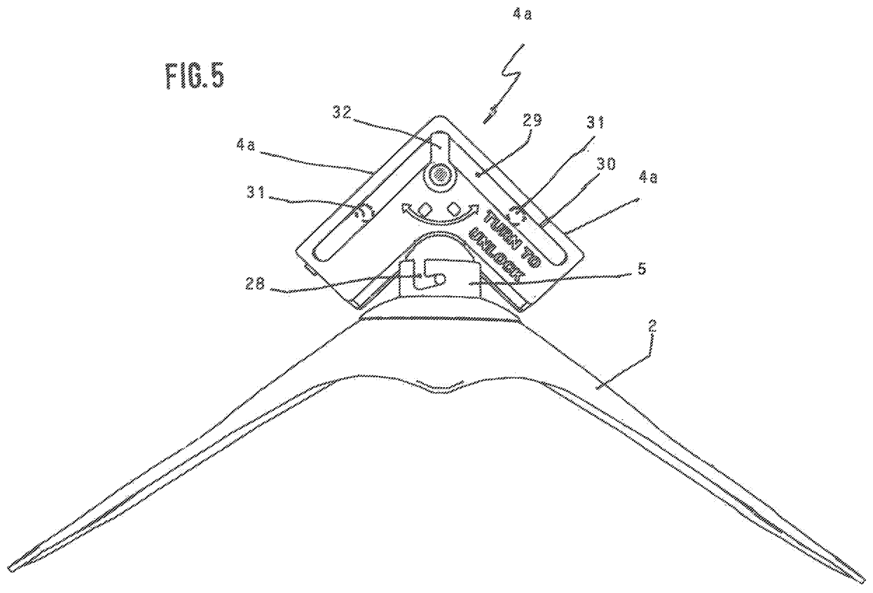

FIG. 5 shows a top view of a variation of the arrangement according to FIG. 1 with an angle-shaped housing;

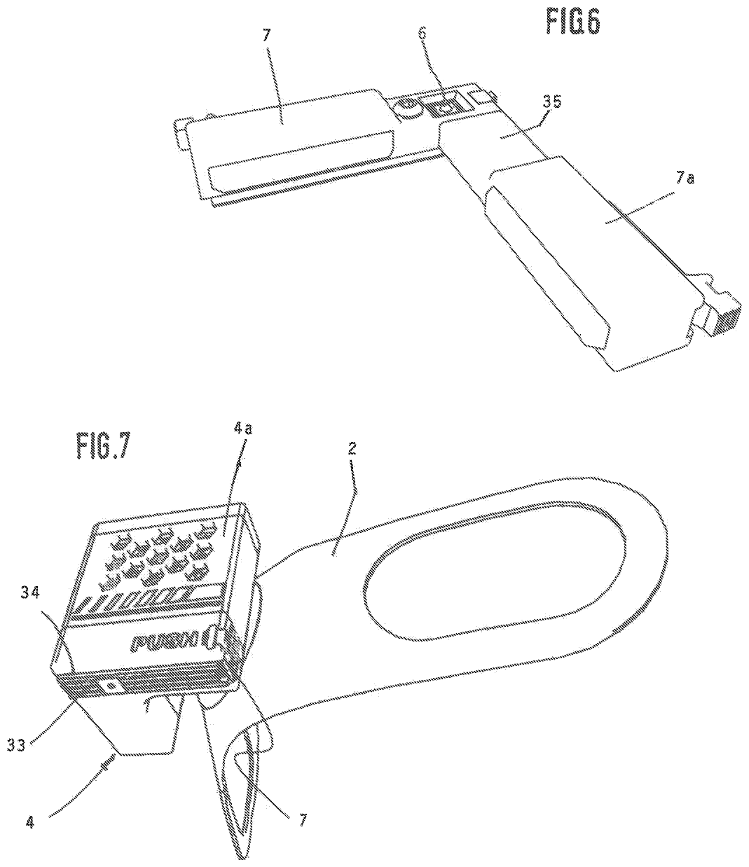

FIG. 6 shows an example of a double slide arrangement;

FIG. 7 shows a perspective view of the arrangement according to FIG. 5 from diagonally below and

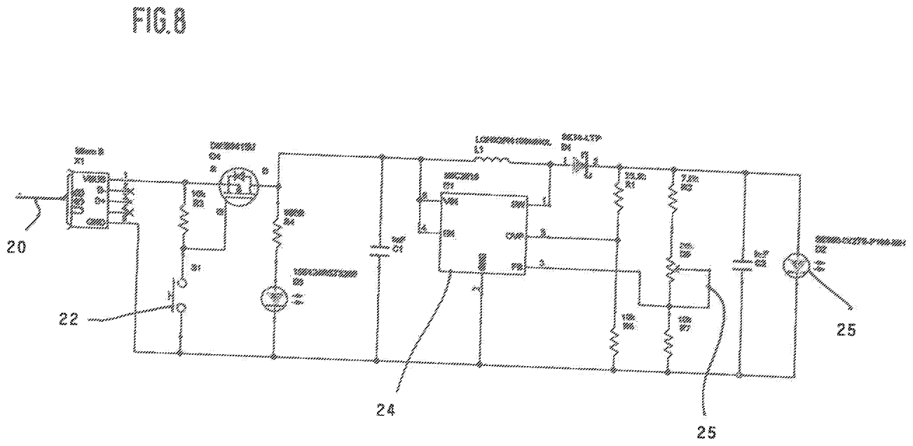

FIG. 8 is an example of an electronic circuit associated with the UV light source.

The respiratory protection mask 1 underlying FIG. 1 contains a face mask part 2 to be worn in front of the wearers face covering mouth and nose and further a device 3 associated with the face mask part 2, with a housing 4 preferably detachably attachable to the face mask part 2. The face mask part 2 essentially consists of textile material, preferably of neoprene or a material exhibiting similar properties, and is provided with a connecting piece 5 to which the housing 4 is detachably connectable. For securing reasons, provision may expediently be made for a bayonet fitting. The internal area of the housing 4 is designed as an air radiation chamber for disinfecting and/or sterilizing the air passing through and possesses an input communicating with the environment and an output communicating with the internal area of the face mask part 2.

Accordingly, the air irradiation chamber of the housing 4 is arranged upstream of the internal area of the lace mask part 2. The air inhaled by the wearer passes through the air irradiation chamber 16 of the housing 4 before it reaches the internal area of the face mask part 2. The air reaching the internal area of the face mask part 2 may expediently be filtered for the purpose of removing solids. For this purpose, a suitable solid matter filter may be arranged in the region of the connecting piece 5 and/or the housing 4, preferably in the region of the output and/or input of the housing 4. Advantageously, the said filter may consist of an aerogel. In simple cases, however, it is also conceivable to do without such filters. In the case a filter is provided, it will advantageously continuously be disinfected.

The air passing through the air irradiation chamber 16 of the housing 4 will be irradiated with germ-killing UV light and thus disinfected and/or sterilized. The air inhaled by the wearer of the present face mask part 1 is thus sterile, and, if a filter is provided, also free from solid particles. To ensure permanent disinfection of a provided filter, it may also be illuminated with germ-killing UV light. In order to treat the air in the internal area of the housing 4 designed as air irradiation chamber 16 with UV light, so-called UVC light is used which may have a wave length in the range of 100 nm to 300 nm, preferably in the range of 250 nm to 260 nm, in particular of 254 nm. To generate the UV radiation for the treatment of the air, provision is made for at least one LED light source 6, preferably designed as UVC-LED, whose light may be radiated in the internal area of the housing 4.

FIG. 2 is a schematic view of such a LED light source 6 arranged on a slide 7 which is detachably receivable in an associated frame 8 of the housing 4, such frame 8, in its internal area, being open towards the internal area of the housing 4, so that the said internal area can be supplied with the UV light of the LED light source 6. The slide 7 is insertable in the frame 8 and can be pulled out thereof. FIG. 1 shows the slide 7 inserted in the associated frame 8, in FIG. 2 it is pulled out. The slide 7, in inserted position, is detachably attached in the frame 8 by means of a suitable snap-on device. In order to release the snap-on connection, the slide 7 may be provided with an unlocking element 9, as is indicated in FIG. 2, which may be expediently designed as a manually actuatable unlocking button. For the formation of the snap-on device, provision may be made for snap-on elements in mutual engagement in the region of the slide 7 and the frame 8. In the example illustrated, the unlocking element 9, for this purpose, is provided with latching teeth 10 overlapping associated counter element in snap-on position.

In order to obtain a high degree of functional safety, the slide 7 may be equipped with a push button 22, indicated in FIG. 2, which can switch on and switch off the LED light source 6. The push button is arranged in such a manner that it can be actuated to switch on the LED light source 6 by an associated element provided in the region of the socket 8 only when the slide 7 is entirely inserted in the socket 8 which likewise increases functional safety. To further increase functional safety, provision may also be made for an operating display device 23, for example formed by a control lamp, indicated in FIG. 2, and in this example, associated with the slide 7. Such control lamp may advantageously be arranged in a manner that it can be seen by the wearer of the respiratory protection mask.

The slide 7 possesses, as is clearly illustrated in FIG. 2, a bulge provided in the rear in insert direction, associated with the unlocking element 9, from which protrudes a flat, longish, latch shaped appendage 12 on which the LED light source 6 is received. The housing-side frame 8 associated with the slide 7 as mentioned earlier is open towards the housing 4. Correspondingly, the LED light source 6 is arranged on the slide 7 in such a manner that, when the slide 7 is inserted in the frame 8, it is located in a region of the frame 8 which is open towards the internal area of the housing 4, and thus can irradiate the internal area of the housing 4 in order to disinfect the air passing through.

As it will be evident from FIG. 3, the housing 4 which, to facilitate manufacture, may comprise two detachably mutually attachable halves 4a, 4b possesses a region in the shape of a cylinder section and an appendage 14 adjoining its jacket 13, in the fashion of a secant, comprising the frame 8. The latter, as can be seen in FIG. 3, is designed as a recess of the appendage 14, intersected with the air irradiation chamber 16 formed by the internal area of the housing 4, comprising guide surfaces 15 associated with the slide 7 and exhibiting a clear configuration adapted to the external configuration of the slide 7.

The LED light source 6 of the slide 7 is placed in a region of the slide 7 reaching the recess of the appendage 14 forming the frame 8 intersected with the air irradiation chamber 16 when the slide 7 is inserted in the frame 8. The input and output of the housing 4 is associated with the mutually opposite end faces of the housing 4. Correspondingly, the two housing halves 4a, 4b comprise the input communicating with the environment or the output communicating with the internal area of the face mask part 2.

Since the UV radiation generated in the air irradiation chamber 16 may be dangerous for the human eye and skin, the said UV radiation will be screened towards the environment and the internal area of the face mask part 2. For this purpose, as is further illustrated in FIG. 3, slat arrangements with parallel, mutually spaced slats 17, which are tilted towards the housing axis, are associated with the input and output of the housing 4. The slats 17 associated with the input provided in the region of the housing half 4a may be provided in the region of the outer side of the housing. The slats 17 associated with the output provided in the region of the other housing half 4b are expediently provided in the region of the internal side of the housing, so that a collision with the connecting pieces 5 or with the installations in the form of filters etc. existing in the region of the connecting piece 5 of the face mask part 2 is prevented.

In order to increase the UV radiation generated in the air irradiation chamber 16, the internal surface 18 of the air irradiation chamber 16 may be expediently covered with mirrors. For this purpose, the internal surface of the housing 4, which may be manufactured as a plastics part, may expediently be provided with a reflecting metal coating in the region of its internal surface and simultaneously be screened against the radiation in the air irradiation chamber 16 as well as protected against ageing. It would very well be conceivable that the entire housing 4 consists of a reflecting material. Expediently, to obtain the aforesaid reflecting properties, provision may be made for a material possessing anti-bacterial properties at the same time, such as for example silver or/and at least a silver compound.

Expediently, the UV light generated in the air radiation chamber 16 is short wave UVC light with a wave length ranging from 210 nm to 300 nm, preferably 250 nm to 260 nm, in particular 254 nm, with the latter being particularly aggressive and correspondently highly suitable for the intended purpose. Thus, the UV light source 6 is correspondingly designed as UVC LED emitting the said UVC light, with a forward voltage of a 5V as well as a radiation power of at least 100 mW at the at the current intensity resulting from the corresponding forward voltage may be provided, Expediently, the LED light source 6 is designed in such a manner that it has a large radiation angle of approx. 120°, so that the air irradiation chamber 16 is well illuminated and that, as previously mentioned, it also illuminates a possibly provided filter.

The LED light source 6 has to be supplied with electricity. For this purpose, the slide 7, in accordance with FIG. 2 and FIG. 3, in the region of a bulge 11 provided in the rear section of the slide 7 in insert direction, is provided with a bushing 19 which is electrically conducting connected with each LED light source 6 received on the slide, such bushing 9 being connectable to a portable energy source 21 formed via a plug-in cable 20. The said energy source 21 may expediently be designed as a so-called electronic power bank which is connectable to the bushing 19 by means of an USB cable. Expediently, provision may be made in the region of the portable housing of the power bank for a display device associated with the LED light source 6 which can easily be seen by the user. Correspondingly, the bushing 19 is expediently designed as a USB bushing and the cable 20 is designed as a USB cable. The control of the UV LED and/or UVC LED forming the LED light source 6 is expediently effected by pulse width modulation of an increasing and decreasing voltage in the form of a saw tooth voltage etc. Alternatively, provision may be made for a circuit designed as a constant electricity source. Such an indirect control will counteract overheating of the said LEDS. As a consequence of direct control the said LEDS may become very hot, so that cooling by means of cooling bodies, etc. may be required.

The above-mentioned energy source 21 supplies direct current of a certain strength. To adjust the requirements of the LED light source 6 consuming current, the electric circuit, as is shown in FIG. 8, associated with the said LED light source 6, may be provided with a DC/DC switching regulator 24. Expediently, the latter is designed as a so-called DC/DC set-up switching regulator which converts the d.c. voltage supplied by the energy source to a d.c. voltage with a higher voltage level. Thus, a comparatively low input voltage may be increased to a usually higher and non-constant forward voltage of the LED light source 6 designed as UVC-LED. The desirable radiation power of each LED light source 6 can be set via the forward voltage, which may reach 100 mW. To avoid an overload of the LED light source 6 the DC/DC step up switching regulator 24 may expediently be provided with a programmable overvoltage protector. Expediently, the circuit associated with the LED light source 6, as is further indicated in FIG. 8, may be equipped with a potentiometer 25 by which the forward voltage is set at the manufacturing plant preferably. From the circuit according to FIG. 8 follows the integration of the push button 22 and the operating display device 23.

Expediently, the LED light source 6 in SMD design may be arranged on the slide 7. Correspondingly, the flap shaped tongue of the slide 7 practically acts as a circuit board wired with the bushing 19 on which the LED light source 6 may be openly arranged. The LED light source 6 comprises at least one LED. An arrangement with several LEDs is likewise conceivable.

FIG. 5 is based on an alternative to FIG. 1 with an angle-shaped design of the housing. The function, however, is identical to the above-mentioned function. Identical parts are thus allocated the same reference numerals. In both alternatives the same functional components may be provided, even if not illustrated. The angle-shaped housing 4 according to FIG. 5 is designed following the human facial symmetry. Thus, the end faces 4a are expediently positioned towards each other with an angle between 90° and 106°. The housing 4, which is detachable from the face mask part 2 and/or its connection piece 5, in this example, is detachably attachable by means of a bayonet fitting 28 resulting in easy handling when the mask is put on and taken off.

The angle-shaped design of the housing 4 practically results in two housing legs adjoining each other at an angle, whereby each leg may be associated with a slide 7. Insofar as a LED light source is provided on each slide 7, an augmented number of existing LED light sources 6 is thus advantageously possible. It would of course likewise be conceivable, as is indicated in FIG. 6, to provide two equally formed slides 7, 7a sewing different purposes, whereby the slide 7 may comprise the LED light source 6 and the other slide 7a may comprise a rechargeable battery 35 or a rechargeable battery module. The two slides in inserted condition, as is indicated in FIG. 6, may be electrically conducting connected with each other, advantageously via a mutual plug-in connection. The plug 7a associated with the rechargeable battery may comprise a connection expediently designed as a USB plug for a charging cable leading to a suitable charge station. It would also be conceivable in a particular advantageous manner to connect the rechargeable battery 35 to a current source via the electrically conducting LED source 6 connected therewith and the cable 20, shown in FIG. 4, which can be plugged in the plug 7 associated with the LED light source 6. A similar arrangement with two mutually opposing slides 7, 7a could also be provided in an arrangement as previously described in connection with FIGS. 1-4.

As long as the cable 20 is plugged in, both the LED light source 6 and the rechargeable battery 35 are supplied with electric current, and the rechargeable battery 37 thus charged. As soon as the cable 20 is plugged off the rechargeable battery 35 takes over the electric current supply of the LED light source.

As previously mentioned, the disinfection device provided in the housing 4 may also comprise a filter 29, as is indicated in FIG. 5. The configuration of the filter 29 in this example follows the configuration of the housing 4 and is thus, like the housing 4, angle-shaped. The filter associated with the disinfection device, in this example, the angle-shaped filter 29, may expediently consist of an aerogel. The filter 29 practically is a firm body which is insertable in a corresponding recess 30 of the housing 4 and/or withdrawable therefrom. Expediently, the filter 29 may be designed as an ejection body which in the region of the recess 30 receiving the latter is associated with ejection springs 31, and which can be locked by means of a rotary lever 32 provided on the housing side and rotatably mounted in the area adjacent to the recess 30. FIG. 5 is based on the locked position in which the rotary lever 32 holds the filter 29, impinged with the force of the ejection springs 31 in the direction of ejection against the force of the ejection 31 in position in the associated recess opening 30. By turning the rotary lever 32 corresponding to the rotary arrow indicated in FIG. 5 the filter 29 is released and can be ejected by effect of the ejection springs 31. Expediently, the filter 29 and the LED source 6 provided to generate germ-killing UV light are positioned towards each other in such a manner that also the filter material is continually illuminated with UV light and thus continually disinfected.

The arrangement contained in the disinfection device for generating UV light may, as previously described, give off a considerable amount of heat. in order to avoid overheating, a cooling body may be associated with the UV light generating device received on the slide. For this purpose, as is indicated in FIG. 6, the slide 7 may be provided with a cooling body 33 which is associated with a housing-side window 34 provided in the region of the socket associated with the slide 7, through which the cooling body 33 is in contact with the surrounding and expediently at least partly may protrude from the housing 4, which particularly encourages the heat exchange with the surrounding.

In addition or alternatively at least part of the warmth given off by the cooling device may be provided to dry the filter 29. For this purpose, provision may be made for airing channels in the housing 4, which are associated with the filter 29. The inlet openings provided in the region of the front ends 4a of the housing 4 may of course be associated with slats for light protection, not shown in detail in FIG. 7, or with other shielding devices suitable for this purpose.

1. A respiratory protection mask with a face mask part (2) and, associated with the latter, an air disinfection device (3) comprising a housing (4) exhibiting an input communicating with the environment, and further an output communicating with the internal area of the face mask part (2) and whose internal area is designed as an air irradiation chamber (16) provided with at least one LED light source (6) emitting UV light, wherein the LED light source (6) is arranged on a slide (7) which is detachably receivable in an associated frame (8) provided on the housing side and open towards the air irradiation chamber (16).

2. A respiratory protection mask in accordance with claim 1, wherein the slide (7) is detachably attachable in the frame (8) by means of an associated snap-on device and has a manually controllable unlocking element (9) attached to the said slide (7).

3. A respiratory protection mask in accordance with claim 2, wherein the slide (7) is provided with a longish flap (12) protruding from a back side bulge (11) on which flap (12) the LED light source (6) in SMD design is received.

4. A respiratory protection mask in accordance with claim 1, wherein the housing (4) preferably consisting of plastics is fitted with a region in the shape of a cylinder section and exhibits an appendage (14) adjoining its jacket (13) in the fashion of a secant, comprising the frame (8), and further that the input and the output of the air irradiation chamber (16) are associated with the mutually opposite end faces of the housing (4).

5. A respiratory protection mask in accordance with claim 1, wherein the frame (8) is designed as a recess of the appendage (14) intersected with the air irradiation chamber (16), such frame (8) comprising guide surfaces (15) associated with the slide (7) and exhibiting a clear configuration adapted to the configuration of the slide (7), with the LED light source (6) being placed in a region of the slide (7) reaching the region of the recess intersected with the air irradiation chamber (16).

6. A respiratory protection mask in accordance with claim 1, wherein the housing (4) is provided with two mutually attachable halves (4a, 4b), each of them comprising a housing end face.

7. A respiratory protection mask in accordance with claim 1, wherein associated with the input and output of the housing (4) are mutually spaced parallel slats (17) which are tilted towards the housing axis and the slats (17) associated with the input are provided in the region of the outer housing side and the slats (17) associated with the output are provided in the region of the internal housing side.

8. A respiratory protection mask in accordance with claim 1, wherein each LED light source (6) is designed as UVC light with a wave length in the range of 250 nm to 260 nm, in particular as UVC LED emitting 254 nm.

9. A respiratory protection mask in accordance with claim 8, wherein each LED light source (6) has a forward voltage of ≥5V as well as a radiation power of 100 mW at the current intensity resulting from the corresponding forward voltage and a large radiation angle in the region of 120°.

10. A respiratory protection mask in accordance with claim 1, wherein the internal surface (18) of the air irradiation chamber (16) is covered with mirrors, and in the case of a housing (6) consisting of plastics, the internal surface (18) is prodded with a reflecting metal coating.

11. A respiratory protection mask in accordance with claim 1, wherein the slide (7) in the region of a bulge (11) provided in the rear section of the slide (7) in insert direction is provided with a bushing (19) which is electrically conducting connected with each LED light source (6) received on the slide, such bushing (19) being connectable to a portable energy source (21) by means of a plug-in cable (20), such portable energy source (21) containing a battery and/or being connectable with external current sources.

12. A respiratory protection mask in accordance with claim 11, wherein the bushing (17) is designed as an USB bushing which is connectable to a portable power bank forming the energy source (21) via the cable (20) designed as an USB cable and the LED light source (6) is controllable by a circuit designed as a pulse width modulation or constant power source of the power bank forming the energy source (21).

13. A respiratory protection mask in accordance with claim 1, wherein the housing (4) is of angular design, with the end faces (4a) being in contact with each other at an angle of 90°-106°.

14. A respiratory protection mask in accordance with claim 1, wherein the air disinfection device (3) is associated with at least one filter (29) and that the filter (29) and the LED light source (6) are positioned towards each another in such a manner that the UV light also shines on the filter (29).

15. A respiratory protection mask in accordance with claim 14, wherein the filter (29) consists of an aero gel.

16. A respiratory protection mask in accordance with claim 14, wherein the filter (29) whose geometry follows the geometry of the housing (4) is receivable in a housing side recess (30) and lockable against the force of ejection springs (31) acting on the rotary lever by means of a rotary lever 32 rotatably mounted on the housing side.

17. A respiratory protection mask in accordance with claim 1, wherein the LED light source (6) is associated with a cooling device which comprises a cooling body (23) received on the slide (7), such cooling body (23) being associated with an opening (34) provided on the housing side.

18. A respiratory protection mask in accordance with claim 17, wherein at least a part of the warmth from the LED light source (6) is provided for drying the filter (29) which is associated with airing channels provided on the housing side.

19. A respiratory protection mask in accordance with claim 1, wherein the slide 7 is associated with a push button (22) with the aid of which a LED light source (6) can be switched on and which is arranged in such a fashion that it is actuated only when the slide (7) is moved in the correct position, and the LED light source (6) is associated with an operating display device (23).

20. A respiratory protection mask in accordance with claim 1, wherein the LED light source (6) is associated with a potentiometer (25) for setting the forward voltage and a DC/DC step-up switching regulator (24) which comprises a programmable overvoltage protection.

21. A respiratory protection mask in accordance with claim 10, wherein the material forming the mirrors covering the internal surface (18) of the irradiation chamber (16) possesses antibacterial properties.

22. A respiratory protection mask in accordance with claim 1, wherein the housing (4) is attachable to the face mask part (2) or to its connecting piece (5) by means of a bayonet fitting (5).

23. A respiratory protection mask in accordance with claim 1, wherein the face mask part (2) consists of neoprene.

24. A respiratory protection mask in accordance with claim 1, wherein the slide (7) containing the LED light source (6) is associated with at least a further slide (7a) containing a rechargeable battery (35) or a rechargeable battery module, with both slides being insertable in an associated socket from different sides which is provided on the housing side, and with their front end regions in the direction of insertion being engageable in electrically conductive engagement.

25. A respiratory protection mask comprising a face mask part (2) and, associated with the latter, an air disinfection device (3) comprising a housing (4) exhibiting an input communicating with the surrounding, and an output communicating with the internal area of the face mask part (2) and whose internal area is designed as an air irradiation chamber (16) provided with at least one light source (6) emitting UV light, and further comprising at least one filter (29) associated with the air disinfection device (3) to filter the air to be treated with UV light streaming through the internal area of the air disinfection device (3), wherein the filter (2) and the light source (6) are positioned towards each other in such a manner that the UV light also shines on the filter (29).
